(19) United States
(12) Patent Application Publication
(10) Pub. No.: US 2020/0289093 A1
(43) Pub. Date: Sep. 17, 2020

(54) ULTRASOUND TRANSDUCER ASSEMBLY HAVING LOW VISCOSITY KERF FILL MATERIAL

(71) Applicant: EchoNous, Inc., Redmond, WA (US)

(72) Inventor: David Nelson, Redmond, WA (US)

(21) Appl. No.: 16/815,421

(22) Filed: Mar. 11, 2020

Related U.S. Application Data
(60) Provisional application No. 62/819,010, filed on Mar. 15, 2019.

(51) Int. Cl. A61B 8/00; B29C 65/48

(52) U.S. Cl. CPC A61B 8/4494; B29L 2031/752; A61B 2562/12; B29C 65/48

(57) ABSTRACT

Ultrasound transducer assemblies and methods include a kerf fill material that substantially fills kerfs between adjacent transducer elements. In at least one embodiment, an ultrasound transducer assembly includes a plurality of transducer elements and a plurality of kerfs. Each of the kerfs is disposed between adjacent ones of the transducer elements. A kerf fill material is disposed in the plurality of kerfs. The kerf fill material includes a first material having a first viscosity and a solvent that reduces the first viscosity of the kerf fill material to a second viscosity that is less than the first viscosity. The kerf fill material may include a mixture of a silicone and a volatile methylsiloxane (VMS) fluid.

### BACKGROUND

### Technical Field

The present disclosure pertains to ultrasound transducer assemblies and methods, and more particularly to ultrasound transducer assemblies and methods having a kerf fill material in kerfs that extend between adjacent transducer elements.

### Description of the Related Art

Diagnostic ultrasound transducer assemblies typically include a plurality of diced transducer elements arranged along an azimuth axis. The transducer assemblies may be included in a device, such as an ultrasound probe, and are used to transmit and receive ultrasonic energy to produce a meaningful image of a targeted biological structure. The diced transducer elements typically include a piezoelectric material, one or more acoustic matching layers, an acoustic lens, and a backing structure. The spaces or gaps between adjacent transducer elements are generally referred to as kerfs.

It is often desirable to provide some mechanical or acoustic isolation between adjacent elements, for example, to reduce crosstalk and improve directivity of the transducer elements in the ultrasound transducer assembly. One method to obtain inter-element isolation (e.g., isolation between adjacent transducer elements) is to leave the kerfs empty, which may be referred to as air kerfs. However, such air kerf architectures generally offer no damping or constraint for lateral vibrations between the adjacent transducer elements, and the impulse response of such ultrasound transducer assemblies will therefore be compromised.

An alternative to air kerfs is to fill the kerfs with some type of kerf fill material. For example, a kerf fill material may be utilized that is designed to constrain or attenuate lateral modes; however, such kerf fill materials may contribute to excessive crosstalk due to a stiffness of the kerf fill material.

### BRIEF SUMMARY

The present disclosure, in part, addresses a desire for improved ultrasound transducer assemblies, in which kerfs between adjacent transducer elements may be more completely filled, and in which an ultrasound lens may be more securely attached, than in conventional designs.

In at least one embodiment, an ultrasound transducer assembly is provided that includes a plurality of transducer elements, a plurality of kerfs respectively disposed between adjacent transducer elements of the plurality of transducer elements, and a kerf fill material in the plurality of kerfs. The kerf fill material includes a first material having a first viscosity and a solvent that reduces the first viscosity of the kerf fill material to a second viscosity that is less than the first viscosity.

In another embodiment, the present disclosure provides a method that includes forming a plurality of kerfs in an ultrasound transducer assembly by dicing through a matching layer and a transducer layer. The kerfs are filled with a kerf fill material, and the kerf fill material includes a mixture of a volatile methylsiloxane (VMS) fluid and at least one of a room temperature vulcanizing (RTV) silicone, an acetoxy, or neutral cure silicone. The method further includes covering a surface of the matching layer with the kerf fill material, and adhesively attaching, by the kerf fill material, an ultrasound lens to the ultrasound transducer assembly.

In another embodiment, an ultrasound probe is provided that includes a housing and an ultrasound transducer assembly that is at least partially enclosed within the housing. The ultrasound transducer assembly includes a plurality of transducer elements on the acoustic backing, at least one matching layer on the plurality of transducer elements, a plurality of kerfs extending in a first direction through the at least one matching layer and at least partially into the acoustic backing, and a kerf fill material in the plurality of kerfs. The kerfs in the plurality of kerfs extend in a second direction between adjacent transducer elements of the plurality of transducer elements, and the second direction is transverse to the first direction. The kerf fill material includes a mixture of a volatile methylsiloxane (VMS) fluid and at least one of a room temperature vulcanizing (RTV) silicone, an acetoxy, or neutral cure silicone.

### BRIEF DESCRIPTION OF THE SEVERAL VIEWS OF THE DRAWINGS

FIG. 1 is a cross-sectional view of an ultrasound transducer assembly, which may be a conventional ultrasound transducer assembly.

FIG. 2 is a perspective view illustrating an ultrasound probe including an ultrasound transducer assembly, in accordance with one or more embodiments of the present disclosure.

FIG. 3 is a cross-sectional view of a transducer assembly taken along the line 3-3 shown in FIG. 2, in accordance with one or more embodiments of the present disclosure.

FIGS. 4 through 7 are cross-sectional views illustrating a method of manufacturing an ultrasound transducer assembly, in accordance with one or more embodiments of the present disclosure.

### DETAILED DESCRIPTION

An ultrasound transducer assembly may include an acoustic backing, a plurality of piezoelectric transducer elements on the acoustic backing, and one or more matching layers on the transducer elements. A plurality of kerfs extends through the matching layers and separate adjacent transducer elements from one another. The kerfs are filled with a kerf fill material that includes a first material, such as RTV silicone, and a solvent such as a volatile methylsiloxane (VMS) fluid that reduces the viscosity of the kerf fill material.

By reducing the viscosity of the kerf fill material, more complete filling of the kerfs may be achieved. Additionally, the reduced-viscosity kerf fill material facilitates inclusion of one or more additives into the kerf fill material while maintaining a viscosity suitable to completely fill the kerfs. Such additives may be included to alter properties of the kerf fill material, which may be selected depending on a particular application, kerf geometry, or desired operating frequency of the ultrasound transducer. The additives may include powders, microparticles, microspheres or the like, which may alter properties of the kerf fill material such as density, viscosity, thermal conductivity, coefficient of thermal expansion (CTE), acoustic attenuation, or stiffness.

In various embodiments, the kerf fill materials provided herein may be provided on an outer surface of a matching layer of an ultrasound transducer assembly, in addition to being provided in the kerfs. In such embodiments, the kerf fill material may be utilized to adhesively attach an ultrasound lens to the outer matching layer.

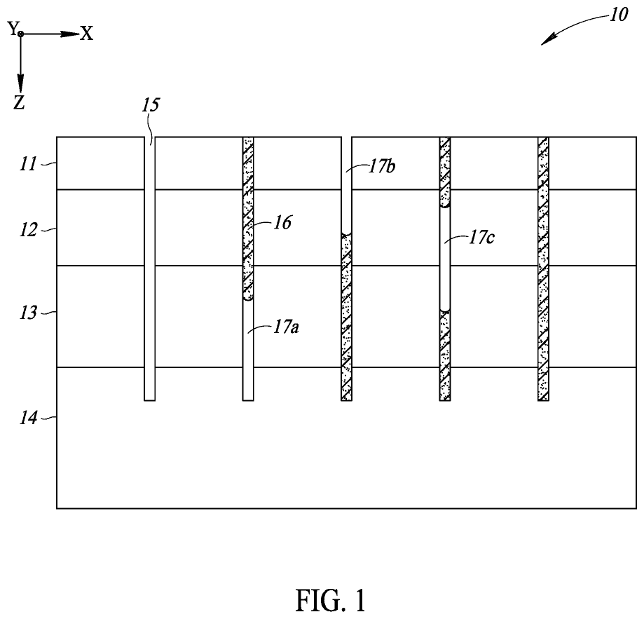

FIG. 1 is a cross-sectional view of an ultrasound transducer assembly 10, which may be a conventional ultrasound transducer assembly. The x-axis represents the azimuth plane, the y-axis represents the elevational plane, and the z-axis represents depth.

The ultrasound transducer assembly 10 includes an acoustic backing 14, a plurality of transducer elements 13, a first matching layer 12, and a second matching layer 11. A plurality of kerfs 15 physically separates the individual transducer elements 13, as well as regions of the first and second matching layers 12, 11 on the transducer elements 13.

A kerf fill material 16 is provided within the kerfs 15. However, as shown in FIG. 1, the kerf fill material 16 may not completely fill the kerfs 15. Instead, air voids 17a, 17b, 17c are present in at least some of the kerfs 15. The presence of the air voids 17a, 17b, 17c indicates incomplete filling of the kerfs 15. This may be caused by various factors, including, for example, the kerfs 15 having a width that is too narrow to be suitably filled by the kerf fill material 16 and/or the kerf fill material 16 having a viscosity that is too high to suitably fill the kerfs 15.

For example, a two-part tin, or platinum curing RTV silicone may be utilized as the kerf fill material 16. However, typical RTV silicone materials (including, for example, RTV664 and RTV630) generally have a viscosity that is greater than about 100,000 centipoise (cps) and may be greater than about 150,000 cps. This relatively high viscosity can impede the ability of the kerf fill material 16 to consistently and repeatably fill the kerfs 15, particularly for transducer assemblies having relatively narrow kerf widths and/or relatively long depths. This incomplete kerf filling may contribute to an impulse response with greater variability and reduced performance.

Accordingly, for ultrasound transducer assemblies in which the kerf fill material 16 results in partially filled kerfs 15 (e.g., including air voids 17a, 17b, 17c), excessive variability and unpredictable performance may result. The partially filled kerfs 15 can be a result of very narrow kerfs, viscous kerf fill material (e.g., having a viscosity greater than about 100,000 cps), inability to properly degas the kerf fill material 16, or any combination of the aforementioned conditions.

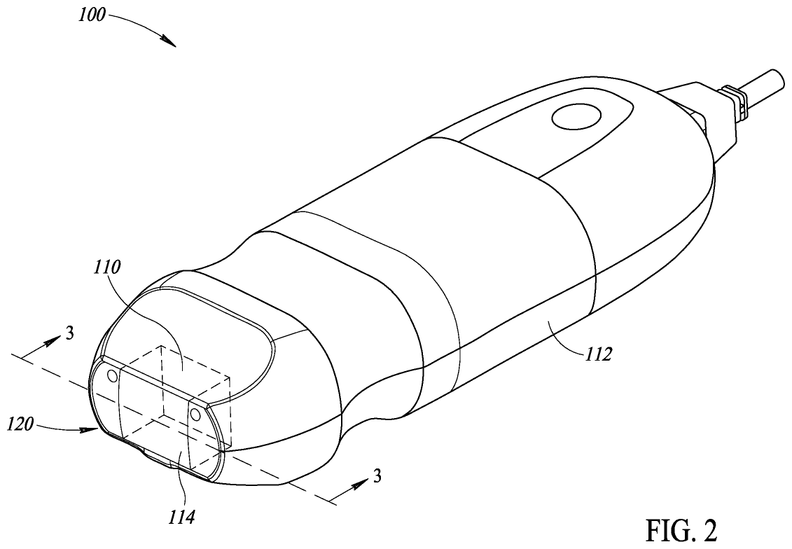

FIG. 2 is a perspective view illustrating an ultrasound probe 100 including an ultrasound transducer assembly 110, in accordance with one or more embodiments of the present disclosure.

The probe 100 includes a housing 112, which forms an external portion of the probe 100. The housing 112 surrounds internal electronic components and/or circuitry of the probe 100, including, for example, electronics such as driving circuitry, processing circuitry, oscillators, beamforming circuitry, filtering circuitry, and the like. The housing 112 may be formed to surround or at least partially surround externally located portions of the probe 100, such as a sensor face 120, and may be a sealed housing, such that moisture, liquid or other fluids are prevented from entering the housing 112. The housing 112 may be formed of any suitable materials, and in some embodiments, the housing 112 is formed of a plastic material. The housing 112 may be formed of a single piece (e.g., a single material that is molded surrounding the internal components) or may be formed of two or more pieces (e.g., upper and lower halves) which are bonded or otherwise attached to one another.

The ultrasound transducer assembly 110 is at least partially enclosed within the housing 112. The transducer assembly 110 includes a plurality of transducer elements which are electrically coupled to internal circuitry housed within the probe 100, such as the driving circuitry, processing circuitry, oscillators, beamforming circuitry, filtering circuitry, and the like.

The transducer assembly 110 is configured to transmit an ultrasound signal toward a target structure in a region of interest in the patient, and to receive echo signals returning from the target structure in response to transmission of the ultrasound signal. To that end, the transducer elements of the transducer assembly 110 are capable of transmitting an ultrasound signal and receiving subsequent echo signals. In various embodiments, the transducer elements may be arranged as elements of a phased array. Suitable phased array transducers are known in the art.

The transducer assembly 110 may include a one-dimensional (1D) array or a two-dimensional (2D) array of transducer elements. The transducer array may include piezoelectric ceramics, such as lead zirconate titanate (PZT), single crystal or may be based on microelectromechanical systems (MEMS). For example, in various embodiments, the transducer assembly 110 may include piezoelectric micromachined ultrasonic transducers (PMUT), which are microelectromechanical systems (MEMS)-based piezoelectric ultrasonic transducers, or the transducer assembly 110 may include capacitive micromachined ultrasound transducers (CMUT) in which the energy transduction is provided due to a change in capacitance.

The ultrasound probe 100 may further include an ultrasound lens 114, which may be included as part of the ultrasound transducer assembly 110, and which may form a part of the sensor face 120 of the probe 100. The lens 114 may be any acoustic lens operable to focus a transmitted ultrasound beam from the transducer elements of the ultrasound transducer assembly 110 toward a patient and/or to focus a reflected ultrasound beam from the patient to the transducer elements. The ultrasound lens 114 may have a curved surface shape in some embodiments. The ultrasound lens 114 may have different shapes, depending on a desired application, e.g., a desired operating frequency, or the like. The ultrasound lens 114 may be formed of any suitable material, and in some embodiments, the ultrasound focusing lens 114 is formed of a room-temperature-vulcanizing (RTV) silicone material.

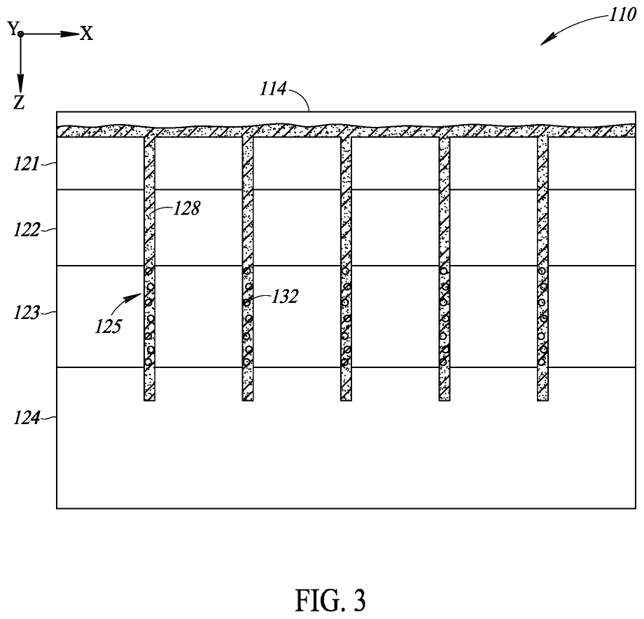

FIG. 3 is a cross-sectional view of the transducer assembly 110 taken along the line 3-3 shown in FIG. 2. The transducer assembly 110 is similar in some respects to the transducer assembly 10 shown and described with respect to FIG. 1; however, kerfs 125 of the transducer assembly 110 are substantially filled by kerf fill material 128, without formation of voids in the kerfs 125 as will be explained in further detail herein.

The transducer assembly 110 includes a plurality of transducer elements 123, which may be, for example, piezoelectric transducer elements. The transducer elements 123 may be formed of any piezoelectric materials, including, for example, lead zirconate titanate (PZT), polyvinylidene fluoride (PVDF), a combination of lead-magnesium-niobate (PMN) and lead titanate (PT) such as a single crystal PMN-PT, or the like.

The transducer elements 123 are formed on an acoustic backing 124. The acoustic backing 124 may be an attenuation element, which reduces or attenuates undesired acoustic reflections and dissipates thermal energy, such as may be generated by vibrations of the transducer elements 123 during operation of the ultrasound probe 100. In some embodiments, the acoustic backing 124 is formed of a composite material, such as a composite including metallic particles and, microspheres in a viscoelastic material, a metal/epoxy composite, a tungsten/vinyl composite, or any other suitable materials.

As shown in FIG. 3, a first matching layer 122 and a second matching layer 121 may be formed on the transducer elements 123, with each of the transducer elements 123 being covered by respective portions of the first and second matching layers 122, 121. The first and second matching layers 122, 121 generally function to increase the transmission of acoustic energy from the high impedance piezoelectric transducer elements 123 to the much lower acoustic impedance of the target to be imaged, such as an organ or other biological structure in a human body. By selecting appropriate matching layer materials and thicknesses, the acoustic impedance can be graded to minimize reflection such that ultrasonic waves emitted by the transducer elements 123 efficiently enter the target for ultrasound imaging.

The first and second matching layers 122, 121 may be formed of any suitable materials having desired acoustical properties, including, for example, any combination of epoxy or resin materials, fillers, and additives.

In some embodiments, the first matching layer 122 may have an acoustic impedance that is greater than an acoustic impedance of the second matching layer 121. While the ultrasound transducer assembly 110 is shown in FIG. 3 as including two matching layers, embodiments of the present disclosure are not limited thereto. In various embodiments, more or fewer than two matching layers may be included in the transducer assembly 110.

The acoustic backing 124, transducer elements 123, and first and second matching layers 122, 121 may be bonded to one another by any suitable technique and/or materials.

A plurality of kerfs 125 extend in the depth direction (e.g., along the z-axis) through the first and second matching layers 122, 121, and laterally separate the transducer elements 123 from one another. The kerfs 125 may extend at least partially into the acoustic backing 124, as shown. In some embodiments, each of the kerfs 125 may extend to a substantially same depth in the transducer assembly 110. In other embodiments, some of the kerfs 125 may extend to different depths in the transducer assembly 110.

The kerfs 125 may have any suitable width (e.g., along the x-axis), which may depend upon various factors, such as a particular application, operational frequency range, or the like of the transducer assembly 110. In some embodiments, each of the kerfs 125 may have a substantially same width. In other embodiments, at least one of the kerfs 125 may have a width that is different from at least one other of the kerfs 125. In some embodiments, the width of the kerfs 125 may be less than about 0.1 mm. In some embodiments, the width of the kerfs 125 may be less than about 50 μm. In some embodiments, the kerfs 125 have a width that is within a range of 20 μm to 40 μm, inclusive.

The kerfs 125 are filled with a kerf fill material 128. In various embodiments, the kerf fill material 128 has attenuation properties to provide suitable isolation between transducer elements 123 while also having a Young's modulus high enough to adequately constrain lateral modes, but not so high as to inhibit transducer element 123 displacement when generating or receiving ultrasonic waves.

In some embodiments, the kerf fill material 128 includes a first material and a solvent which reduces the viscosity of the first material. For example, in some embodiments, the kerf fill material 128 includes a room temperature vulcanizing (RTV) silicone and a solvent which facilitates a viscosity reduction of the RTV silicone. The silicone may be any silicone material, including, for example, a single part (acetoxy or neutral cure) silicone, a two-part (condensation or addition cure) RTV silicone, or an amalgam of a single and two-part silicone system.

In some embodiments, the solvent in the kerf fill material 128 includes one or more siloxanes. In some embodiments, the solvent in the kerf fill material 128 includes volatile methylsiloxane (VMS) fluids, which facilitate a viscosity reduction of the kerf fill material 128, such as RTV silicone. In some embodiments, the kerf fill material 128 has a viscosity less than about 1000 cps (centipoise). In some embodiments, the kerf fill material 128 is an ultra-low viscosity material having a viscosity within a range of about 25 cps to 250 cps, inclusive.

By utilizing a solvent, such as a VMS fluid, in the kerf fill material 128, the kerf fill material 128 may have a viscosity that is significantly reduced as compared to that of a conventional single part or two-part RTV silicone. The solvency of VMS fluids in silicone compounds (e.g., RTV silicone) allows the VMS fluids to serve as a diluent to reduce the silicone compound's viscosity. VMS fluids are available in a range of different vapor pressures, and thus, in various embodiments, the working life and final material porosity of the composite kerf fill material 128 may be tailored as may be desired, for example, depending on a particular application or desired operational characteristics of the transducer assembly 110, dimensions of the transducer assembly 110, dimensions of the kerfs 125, or the like.

As a result of the significant viscosity reduction of the first material (e.g., RTV silicone) in the kerf fill material 128, the first material can be relatively heavily filled with other materials to further alter the properties of the kerf fill material 128. For example, the reduced-viscosity RTV silicone can contain a relatively high concentration of additional materials while retaining a desired low viscosity to completely fill the kerfs 125. In contrast, loading a typical RTV silicone material with a similar concentration of additional materials may increase the viscosity of the RTV silicone material to a point at which it is unsuitable to completely fill the kerfs 125, and instead may result in formation of voids in the kerfs 125.

As shown in FIG. 3, the kerf fill material 128 may include an additive 132, which may be, for example, any material which is added to the first material (e.g., the RTV silicone), and which alters one or more properties or characteristics of the kerf fill material 128. In some embodiments, the additive 132 may alter any of density, viscosity, coefficient of thermal expansion (CTE), acoustic attenuation, thermal conductivity, or stiffness of the kerf fill material 128. The additive 132 includes a material that is different from the first material of the kerf fill material 128.

In various embodiments, the additive 132 may be or include any metallic or metallic oxide powder, polymeric powders, or microparticles such as microspheres. In some embodiments, the additive 132 includes microspheres, which may be any generally spherical microparticles, and which may have a size (e.g., a diameter) between about 1 μm and 1 mm. In some embodiments, the additive 132 includes glass or polymeric microspheres which may decrease density, increase viscosity, and/or reduce CTE of the kerf fill material 128. The additive 132 may include glass microspheres, which may reduce the CTE of the kerf fill material 128. In some embodiments, the additive 132 may include hollow microspheres, which may reduce the density of the kerf fill material 128. Microspheres or finely ground microparticles such as cured silicone can also be included in the kerf fill material 128, in some embodiments, to increase attenuation and reduce inter-element crosstalk (e.g., crosstalk between the transducer elements 123).

In some embodiments, the additive 132 includes a powder, such as a powder including one or more of aluminum nitride (AlN), magnesium oxide (MgO), boron nitride (BN), diamond, or copper, which can be added to the kerf fill material 128 to increase thermal conductivity.

In some embodiments, the additive 132 is included in at least portions of the kerf fill material 128 that is disposed in the kerfs 125. In some embodiments, the additive 132 is uniformly distributed throughout the kerf fill material 128, and the kerf fill material 128 may be a homogenous mixture including the first material (e.g., RTV silicone) and the additive 132. In other embodiments, the additive 132 is non-uniformly distributed in the kerf fill material 128. For example, in some embodiments, the additive 132 may be dispersed in the first material with a concentration gradient, for example, with a concentration that increases along the depth (e.g., z-axis) of the kerfs 125. In some embodiments, the additive 132 may have a concentration that is highest in regions of the kerfs 125 directly between adjacent transducer elements 123. This may provide the particular altered characteristics of the kerf fill material 128 in a focused region between the transducer elements 123, while other portions of the kerf fill material 128 may have a lower concentration of the additive 132 or may be substantially free of the additive 132.

In addition to filling the kerfs 125, the kerf fill material 128 may cover a surface (e.g., the upper surface) of the second matching layer 121, as shown in FIG. 3. The ultrasound lens 114 may be attached to the second matching layer 121 by adhesion provided from the layer of the kerf fill material 128 between the second matching layer 121 and the ultrasound lens 114. In some embodiments, the kerf fill material 128 has a thickness over the upper surface of the second matching layer 121 that is within a range of 0.01 mm to 5 mm. In some embodiments, the thickness of the kerf fill material 128 over the upper surface of the second matching layer 121 is within a range of 0.1 mm to 0.5 mm, which provides enhanced adhesion between the second matching layer 121 and the ultrasound lens 114.

In some embodiments, the ultrasound lens 114 is formed of RTV silicone material, which may be the same or different from the silicone material which may be included as the first material in the kerf fill material 128. In some embodiments, the ultrasound lens 114 is formed of a two-part addition cure RTV silicone. The addition of the solvent (e.g., a VMS fluid) in the composite kerf fill material 128 enhances the adhesion of the kerf fill material 128, thereby improving adhesion of the RTV silicone ultrasound lens 114 to the transducer assembly 110.

Once the transducer assembly 110 has been assembled, e.g., with the ultrasound lens 114 being formed over the outer matching layer (e.g., the second matching layer 121, as shown), the kerf fill material 128 may be cured. In some embodiments, the solvent in the kerf fill material 128 (e.g., the VMS fluid) is liberated during the curing process, which results in a silicone structure with an increased silicon (Si) chain length and lower final Young's modulus and shore A hardness (or lower durometer rating). The lower hardness (e.g., as indicated by a lower durometer rating) of the cured kerf fill material 128 may reduce friction and crosstalk between transducer elements 123 of the transducer assembly 110.

The ultrasound lens 114 may form an outer layer of the transducer assembly 110, and may form an exposed portion of the ultrasound probe 100. For example, the ultrasound lens 114 may be positioned along the sensor face 120 of the ultrasound probe 100, as shown in FIG. 2.

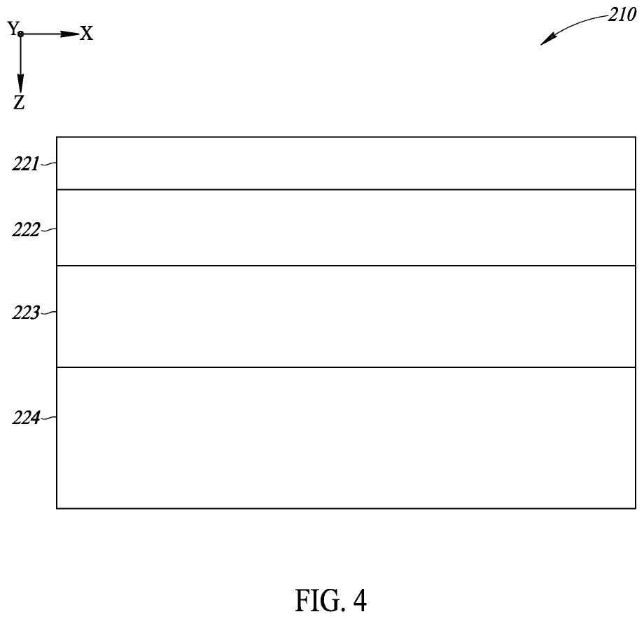

FIGS. 4 through 7 are cross-sectional views illustrating a method of manufacturing an ultrasound transducer assembly, such as the ultrasound transducer assembly 110 shown in FIG. 3, in accordance with one or more embodiments.

As shown in FIG. 4, a method of manufacturing an ultrasound transducer assembly may include forming an ultrasound transducer block 210. The ultrasound transducer block 210 includes an acoustic backing 224, an ultrasound transducer layer 223 on the acoustic backing 224, a first matching layer 222 on the ultrasound transducer layer 223, and a second matching layer 221 on the first matching layer 222.

The acoustic backing 224, ultrasound transducer layer 223, first matching layer 222, and second matching layer 221 may be laminated or bonded to one another by any suitable material and/or technique. For example, in some embodiments, the layers of the ultrasound transducer block 210 may be bonded to one another by one or more adhesives, such as an epoxy.

While the ultrasound transducer block 210 is shown in FIG. 4 as including two matching layers, embodiments of the present disclosure are not limited thereto. In various embodiments, more or fewer than two matching layers may be included in the ultrasound transducer block 210.

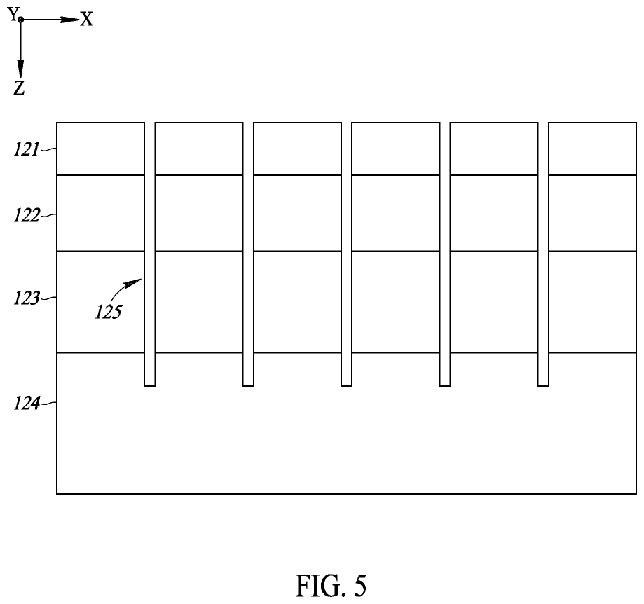

As shown in FIG. 5, the method may further include forming a plurality of kerfs 125 which extend through the second matching layer 221, the first matching layer 222, and the ultrasound transducer layer 223. In some embodiments, the plurality of kerfs 125 extend at least partially into the acoustic backing 224. The kerfs 125 may be formed, for example, by dicing the ultrasound transducer block 210, thereby forming the acoustic backing 124, the transducer elements 123, and separate regions of the first and second matching layers 122, 121 as shown in FIG. 5. The kerfs 125 thus separate the individual transducer elements 123 from one another, and further separate regions or portions of the first and second matching layers 122, 121.

The kerfs 125 may be formed to have any suitable width, e.g., extending between adjacent ones of the transducer elements 123. In some embodiments, the kerfs 125 may be formed to have a width that is within a range of 20 μm to 40 μm, inclusive.

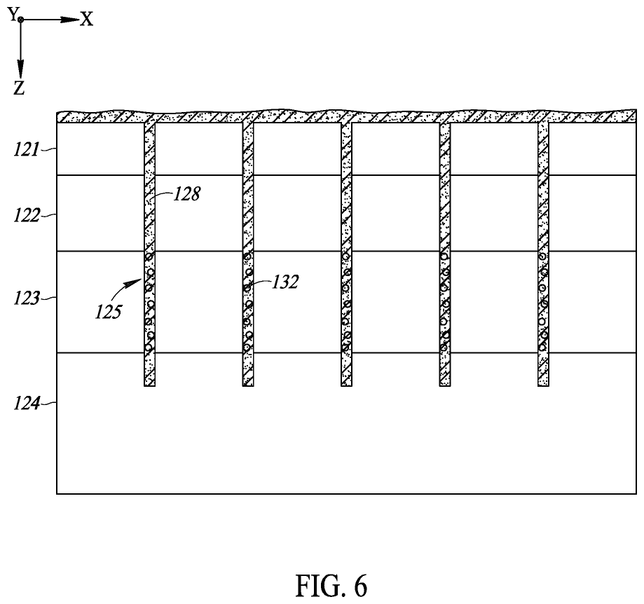

As shown in FIG. 6, the method may further include filling the plurality of kerfs 125 with a kerf fill material 128. The kerf fill material 128 may include a mixture of a first material and a solvent. In some embodiments, the first material of the kerf fill material 128 includes a room temperature vulcanizing (RTV) or single part (acetoxy or neutral cure) silicone, and the solvent includes a volatile methylsiloxane (VMS) fluid. In some embodiments, the kerf fill material 128 may further include one or more additives 132, which may include at least one of a metallic powder, a metallic oxide powder, microparticles, or microspheres.

The kerf fill material 128 may further cover a surface (e.g., an upper surface) of the second matching layer 121, as shown. The kerf fill material 128 may have a thickness on the surface of the second matching layer 121 that is within a range of 0.1 mm to 0.5 mm, inclusive.

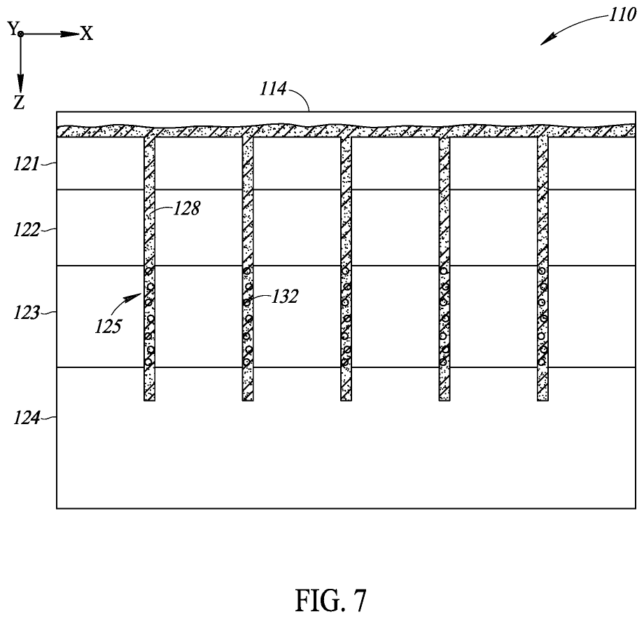

As shown in FIG. 7, the method may further include attaching an ultrasound lens 114 to the second matching layer 121. The ultrasound lens 114 may be adhesively attached to the second matching layer 121, for example, by the kerf fill material 128 that extends between the upper surface of the second matching layer 121 and the ultrasound lens 114.

The ultrasound lens 114 may be formed to have any shape, and in some embodiments, the ultrasound lens 114 is formed to have a curved shape, for example, along an outer surface of the ultrasound lens 114. The ultrasound lens 114 may be formed of any suitable material, and in some embodiments, the ultrasound focusing lens 114 is formed of a room-temperature-vulcanizing (RTV) silicone material.

In various embodiments provided herein, ultrasound transducer assemblies and methods are provided which facilitate improved adhesion of the ultrasound lens 114 to an outer matching layer, such as the second matching layer 121. The improved adhesion is provided, for example, by the kerf fill material 128 which may include a mixture of a silicone and a volatile methylsiloxane (VMS) fluid. The VMS fluid reduces the viscosity of the silicone, which may facilitate consistent and convenient spreading of the kerf fill material 128 over a surface of the second matching layer 121. Moreover, the composite kerf fill material 128 may have improved adhesion properties as compared to a conventional RTV silicone.

Additionally, embodiments of the present disclosure facilitate tailoring of the kerf fill material 128 to have various properties or characteristics depending on a desired application or design of the ultrasound transducer assembly 110. For example, by including an additive 132 in the kerf fill material 128, characteristics such as density, viscosity, coefficient of thermal expansion (CTE), acoustic attenuation, thermal conductivity, or stiffness of the kerf fill material 128 may be altered as may be desired for various applications, kerf geometries, operating frequencies or the like.

Moreover, embodiments of the present disclosure facilitate improved filling of the kerfs 125 in the ultrasound transducer assembly 110. For example, due to the reduced viscosity of the kerf fill material 128, the kerfs 125 may be completely filled, thereby reducing formation of voids in the kerfs which may otherwise occur when filled with a conventional RTV silicone. Additionally, the kerf fill material 128 provided by the present disclosure more consistently and repeatably fills the kerfs 125, thereby reducing variations in the kerf filling processes which otherwise may occur when filled with a conventional RTV silicone which generally yields incomplete and inconsistent filling of the kerfs.

The various embodiments described herein can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An ultrasound transducer assembly, comprising:
a plurality of transducer elements;
a plurality of kerfs respectively disposed between adjacent transducer elements of the plurality of transducer elements; and
a kerf fill material in the plurality of kerfs, the kerf fill material including:
a first material having a first viscosity; and
a solvent that reduces the first viscosity of the kerf fill material to a second viscosity that is less than the first viscosity.

2. The ultrasound transducer assembly of claim 1 wherein the first material includes silicone.

3. The ultrasound transducer assembly of claim 2 wherein the solvent includes a siloxane.

4. The ultrasound transducer assembly of claim 2 wherein the solvent includes a volatile methylsiloxane (VMS) fluid.

5. The ultrasound transducer assembly of claim 1 wherein the second viscosity is within a range of 25 centipoise to 250 centipoise, inclusive.

6. The ultrasound transducer assembly of claim 1 wherein the kerf fill material further includes at least one additive dispersed within the first material, and the at least one additive alters one or more properties of the kerf fill material.

7. The ultrasound transducer assembly of claim 6 wherein the at least one additive includes one or more of a metallic powder, a metallic oxide powder, microparticles, or microspheres.

8. The ultrasound transducer assembly of claim 6 wherein the at least one additive alters one or more of a density, viscosity, coefficient of thermal expansion, acoustic attenuation, thermal conductivity, or stiffness of the kerf fill material.

9. The ultrasound transducer assembly of claim 6 wherein the at least one additive is uniformly distributed in the kerf fill material.

10. The ultrasound transducer assembly of claim 1, further comprising:
an acoustic backing; and
a first matching layer,
wherein the plurality of transducer elements is positioned between the acoustic backing and the matching layer, and the kerfs extend through the first matching layer and at least partially into the acoustic backing.

11. The ultrasound transducer assembly of claim 10, further comprising a second matching layer on the first matching layer, wherein the kerfs further extend through the second matching layer.

12. The ultrasound transducer assembly of claim 11 wherein the kerf fill material covers a surface of the second matching layer.

13. The ultrasound transducer assembly of claim 12, further comprising an ultrasound lens disposed over the surface of the second matching layer, wherein the kerf fill material that covers the surface of the second matching layer adhesively attaches the ultrasound lens to the surface of the second matching layer.

14. The ultrasound transducer assembly of claim 13 wherein the ultrasound lens includes RTV silicone.

15. The ultrasound transducer assembly of claim 12 wherein the kerf fill material has a thickness on the surface of the second matching layer within a range of 0.1 mm to 0.5 mm, inclusive.

16. The ultrasound transducer assembly of claim 10 wherein the kerf fill material further includes at least one additive dispersed within the first material, the at least one additive alters one or more properties of the kerf fill material, and the kerf fill material has a higher concentration of the at least one additive in portions of the kerfs disposed directly between the adjacent transducer elements of the plurality of transducer elements than in one or more other portions of the kerfs.

17. A method, comprising:
forming a plurality of kerfs in an ultrasound transducer assembly by dicing through a matching layer and a transducer layer of the ultrasound transducer assembly;
filling the plurality of kerfs with a kerf fill material, the kerf fill material including a mixture of a volatile methylsiloxane (VMS) fluid and at least one of a room temperature vulcanizing (RTV) silicone, an acetoxy, or neutral cure silicone;
covering a surface of the matching layer with the kerf fill material; and
adhesively attaching, by the kerf fill material, an ultrasound lens to the ultrasound transducer assembly.

18. The method of claim 17 wherein covering the surface of the matching layer with the kerf fill material includes covering the surface of the matching layer with a thickness of the kerf fill material that is within a range of 0.1 mm to 0.5 mm, inclusive.

19. The method of claim 17 wherein the kerf fill material further includes an additive, the additive including at least one of a metallic powder, a metallic oxide powder, microparticles, or microspheres.

20. An ultrasound probe, comprising:
a housing; and
an ultrasound transducer assembly that is at least partially enclosed within the housing, the ultrasound transducer assembly including:
an acoustic backing;
a plurality of transducer elements on the acoustic backing;
at least one matching layer on the plurality of transducer elements;
a plurality of kerfs extending in a first direction through the at least one matching layer and at least partially into the acoustic backing, the kerfs in the plurality of kerfs extending in a second direction between adjacent transducer elements of the plurality of transducer elements, the second direction being transverse to the first direction; and
a kerf fill material in the plurality of kerfs, the kerf fill material including a mixture of a volatile methylsiloxane (VMS) fluid and at least one of a room temperature vulcanizing (RTV) silicone, an acetoxy, or neutral cure silicone.

21. The ultrasound probe of claim 20 wherein the ultrasound transducer assembly further includes an ultrasound lens on the at least one matching layer, and wherein the kerf fill material covers a surface of the at least one matching layer and adhesively attaches the ultrasound lens to the surface of the at least one matching layer.

22. The ultrasound probe of claim 20 wherein the kerf fill material further includes at least one of a metallic powder, a metallic oxide powder, microparticles, or microspheres.
